(19) United States
(12) Patent Application Publication
(10) Pub. No.: US 2026/0263333 A1
(43) Pub. Date: Sep. 10, 2026

(54) COATED COLOURING PARTICLES OF METAL OXIDES AND SUBOXIDES, AND THEIR PREPARATION BY FLAME SPRAY PYROLYSIS

(71) Applicant: L'Oreal, Paris (FR)

(72) Inventors: Valérie Jeanne-Rose, AULNAY-SOUS-BOIS (FR); Henri Samain, CHEVILLY-LARUE (FR); Yiannis Deligiannakis, IOANNINA (GR); Maria Louloudi, IOANNINA (GR)

(73) Assignee: L'Oreal, Paris (FR)

(21) Appl. No.: 18/722,258

(22) PCT Filed: Dec. 20, 2022
(86) PCT No.: PCT/EP2022/087125

§ 371 Date: Jun. 20, 2024

(30) Foreign Application Priority Data
Dec. 21, 2021 (FR) 2114166

(51) Int. Cl. A61K 8/19; A61K 8/25; A61Q 1/02; B01J 10/00; B01J 19/26; C09C 3/06

(52) U.S. Cl. CPC A61K 8/19; A61K 8/25; A61Q 1/025; B01J 10/00; B01J 19/26; C09C 3/063; A61K 2800/621; A61K 2800/651

(57) ABSTRACT

The present invention relates to the use of coated particles of oxide and of suboxide of element M for dyeing and/or making up keratin materials, to a process for the preparation of such coated particles by means of the flame spray pyrolysis technology, to the particles of oxide and of suboxide of element M resulting from such a process, to the coated particles of suboxide of element M and also to the compositions comprising such particles.

The present invention relates to the use of coated particles of oxides and of suboxides of element M for dyeing and/or making up keratin materials, to a process for the preparation of such coated particles by means of the flame spray pyrolysis technology, to the particles of oxides and of suboxides of element M resulting from such a process, to the coated particles of suboxide of element M and also to the compositions comprising such particles.

Metal oxides are used in numerous applications (cosmetics, paints, stains, electronics, rubber, and the like), in particular for their colouring properties.

Currently, the colours of formulations, for example cosmetic formulations, are generally obtained by the mixing of several coloured metal oxides.

However, some oxides exhibit the disadvantage of being particularly unstable over time, which brings about a deterioration in their colouring power, more particularly a deterioration in the intensity and/or in the chromaticity of the colour obtained.

Thus, it may happen that the colours of the formulations change over time and result in colours which are not very intense, dull and/or undesired by the user. It might even be that the differences in stability of the different metal oxides which are mixed disrupt the homogeneity of the colouring mixture and for example bring about migration of oxide to the surface or into the bottom of the formulation. This undesirable effect is referred to as “separation effect”.

It has been envisaged, in order to improve the stability of the metal oxides, to coat the metal oxides with silica, in particular by means of “sol-gel” processes, or also to graft fluorinated compounds to the metal oxides. However, these solutions are not entirely satisfactory. The metal oxides coated with silica by a sol-gel process generally exhibit poorer colouring properties than a non-coated particle. As for the grafting technique, the use of fluorinated compounds can be harmful to the environment and dangerous to the user.

It is also known to use a flame spray pyrolysis technology or FSP method to prepare metal oxide particles.

Flame spray pyrolysis or FSP is a method well known today, which has essentially been developed for the synthesis of ultrafine powders of single or mixed oxides of various metals (e.g. SiO₂, Al₂O₃, B₂O₃, ZrO₂, GeO₂, WO₃, Nb₂O₅, SnO₂, MgO, ZnO), having controlled morphologies, and/or their deposition on various substrates, this being carried out starting from a wide variety of metal precursors, generally in the form of organic or inorganic, preferably inflammable, sprayable liquids; the liquids sprayed into the flame, on being consumed, in particular emit nanoparticles of metal oxides which are projected by the flame itself over these various substrates. The principle of this method has been restated, for example, in the recent (2011) publication by Johnson Matthey entitled “Flame Spray Pyrolysis: a Unique Facility for the Production of Nanopowders”, Platinum Metals Rev., 2011, 55 (2), 149-151. Numerous alternative forms of FSP processes and reactors have also been described, by way of examples, in the patents or patent applications WO 2007/028267 or U.S. Pat. No. 8,182,573, EP 1760043, U.S. Pat. Nos. 5,958,361, 2,268,337, WO 01/36332 or U.S. Pat. No. 6,887,566, WO 2004/005184 or U.S. Pat. No. 7,211,236, WO 2004/056927, WO 2005/103900, WO 2008/049954 or U.S. Pat. No. 8,231,369, WO 2008/019905, US 2009/0123357, US 2009/0126604, US 2010/0055340, WO 2011/020204.

However, the particles prepared according to the known processes by flame spray pyrolysis are not always satisfactory in terms of stability. More particularly, these preparation processes do not make it possible to obtain, easily and in large number, oxides of metals with an intermediate oxidation number or metal suboxides. Furthermore, the oxides of metals with an intermediate oxidation number and metal suboxides prepared according to these known processes are not stable over time and oxidize to give their maximum oxidation number very rapidly on contact with ambient air.

In addition, the particles prepared by flame spray pyrolysis can still be improved, in particular in terms of intensity and of chromaticity of the colour provided.

There thus exists a real need to develop a method for dyeing and making up keratin materials starting from metal oxide particles which is capable of providing keratin materials with an intense and chromatic colouring.

In addition, it is desirable to develop a broad range of these metal oxide particles, exhibiting good stability over time and good colouring properties, in particular in terms of intensity and of chromaticity of the colour conferred. This broad range of particles should make it possible to obtain a rich palette of colours and thus to obtain more varied and more nuanced colourings more faithful to the wishes of the user.

It is thus necessary to develop a process which makes it possible to prepare such particles and more particularly to prepare particles of oxides of metals with an intermediate oxidation number and metal suboxides exhibiting good stability over time and good properties for the dyeing and making up of keratin materials.

These aims are achieved with the present invention, a subject matter of which is in particular the use, for the dyeing and/or making up of keratin materials, in particular of human keratin fibres and the skin, of at least one particle of oxide of element M, in particular of the type of oxide of M-M′ of core/shell structure, comprising:
(i) a core (1) constituted of at least one oxide of element M of formula (I):

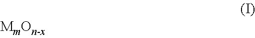

in which:
   M represents an element chosen from the alkali metals of column 1, the alkaline earth metals of column 2 and the elements of columns 3 to 14 of the Periodic Table of the Elements, bismuth, selenium, tellurium and astatine,
   m represents an integer greater than or equal to 1,
   n represents an integer greater than or equal to 1,
   x is equal to 0 or represents a non-integral number greater than 0 and strictly less than n, and
(ii) an upper coating layer (2), covering the surface of said core (1), constituted of at least one compound of formula (II):

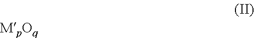

in which:
   M′ is an element, other than M, chosen from selenium and the elements of columns 4, 13 and 14 of the Periodic Table of the Elements,
   p represents an integer greater than or equal to 1,
   q represents an integer greater than or equal to 0.

It has been found that the particles according to the invention make it possible to obtain colourings of keratin materials which are particularly intense and chromatic.

Furthermore, it has been possible to prepare a large number of particles according to the invention, with a large number of different and atypical colours (for example fluorescent colours).

The particles according to the invention have thus made it possible to obtain a rich palette of colours, which makes possible a greater number of colourings and thus a colouring closer to the wish of the user. This also makes it possible to reduce the number of metal oxides to be mixed in order to obtain the desired colour.

In addition, it has been noticed that the coated colouring particles of oxides of element M according to the invention only deteriorate very little over time, this being the case even when they are formulated in an in particular aqueous composition.

In particular, only very little in the way of “separation effects” have been observed with the particles according to the invention.

It has also been observed that the cosmetic make-up compositions comprising coated colouring particles of oxides of element M according to the invention exhibit a good power of masking (for example imperfections of the skin) and also make it possible to obtain colourings which are particularly covering (for example for mascaras).

In addition, as the coated particles of oxides of element M according to the invention do not require a hydrophobic coating, it is possible to use them in a broad formulation spectrum (for example, in entirely aqueous formulations and/or surfactant-free formulations). When the formulations thus obtained end up in water (washbasin drainage, lake or sea), the risk of inappropriate deposit (on the edges of the washbasin, on the walls of the pipes or on rocks) is furthermore reduced.

The invention also relates to a particle of suboxide of element M comprising: (i) a core (1) constituted of at least one oxide of element M of formula (I′):

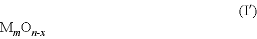

in which:
   M represents an element chosen from the alkali metals of column 1, the alkaline earth metals of column 2 and the elements of columns 3 to 14 of the Periodic Table of the Elements, bismuth, selenium, tellurium and astatine,
   m represents an integer greater than or equal to 1,
   n represents an integer greater than or equal to 1;
   x represents a non-integral number greater than 0 and strictly less than n, and
(ii) an upper coating layer (2), covering the surface of said core (1), constituted of at least one compound of formula (II):

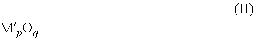

in which:
   M′ is an element, other than M, chosen from selenium and the elements of columns 4, 13 and 14 of the Periodic Table of the Elements,
   p represents an integer greater than or equal to 1,
   q represents an integer greater than or equal to 0.

It has been observed that the particles of suboxide of element M according to the invention are particularly stable over time (i.e., the particles remain in their suboxide state) and make it possible to obtain colourings which are significantly intense, chromatic and atypical.

Another subject-matter of the invention relates to a process for the preparation of such particles of oxide and of suboxide of element M which are coated with an oxide of element M′, in particular of the type of oxide of M-M′ of core/shell structure, comprising at least the following stages:
a. preparing a composition (A) by adding one or more precursors of element M to a combustible solvent or to a mixture of combustible solvents; then
b. forming a flame, in a flame spray pyrolysis device, by injecting the composition (A) and an oxygen-containing gas (G) until aggregates of oxide of element M are obtained; then
c. injecting a composition (B) comprising one or more precursors of element M′ until an upper coating layer (2) constituted of element M′ or of oxide of element M′ is obtained, at the surface of said aggregates of oxide of element M;
   said element M being chosen from the alkali metals of column 1, the alkaline earth metals of column 2 and the elements of columns 3 to 14 of the Periodic Table of the Elements, bismuth, selenium, tellurium and astatine; and said element M′ being other than M and chosen from selenium and the elements of columns 4, 13 and 14 of the Periodic Table of the Elements; and said flame spray pyrolysis device being isolated from the exterior air, so that the amount of oxygen present in said device is controlled.

It has been observed that the process according to the invention makes it possible to obtain particles of oxide and of suboxide of element M which are coated with a layer of inorganic material based on element M′, which are particularly stable over time and exhibit good water resistance.

Furthermore, unlike conventional coating processes, the process according to the invention has the advantage, despite the presence of an upper coating layer, of retaining good intrinsic performance qualities of the core. This is because, due to the specific nature of the upper coating layer, it is possible, for a given particle weight, to reduce the proportion of oxide or of suboxide of element M, without, however, reducing and/or negatively affecting the properties of said oxide or suboxide of element M.

Thus, the process of the invention makes it possible to produce stable particles of oxide and of suboxide of element M, while avoiding the inconveniences due to the increase in the amount of particles which would be conventionally necessary in order to maintain the good colouring properties of the oxide or of the suboxide of element M.

According to another aspect, the invention also relates to a specific flame spray pyrolysis device for the implementation of the preparation process of the invention, comprising a first chamber, a second chamber in communication with or connected fluidically to the first chamber, an injection system comprising a first feed, for example a first tube, emerging in the first chamber and capable of delivering a first composition (A) and a first oxygen-containing gas (G), and a second feed, for example a second tube, emerging in the first chamber and capable of delivering a mixture (P) comprising oxygen and one or more combustible gases, the first and second feeds being separate from one another.

The device further comprises a third feed capable of delivering a second composition (B) comprising one or more precursors of element (M′) into the second chamber.

The first and second chambers of said device are isolated from the external air, so that the amount of oxygen present in said device is controlled, and more preferentially so that the oxygen present in said first and second chambers originates solely from said first gas (G) and optionally from said mixture (P).

For example, in a way which is not at all limiting, the second chamber is coaxial with the first chamber and, for example, positioned in the prolongation of said first chamber.

Advantageously, the first and second feeds are coaxial, the second feed at least partially surrounding the first feed.

According to one embodiment, the first chamber comprises two separate compartments, the first compartment comprising a first opening in which the injection system emerges and a second opening, on the side opposite the first opening, the second compartment at least partially surrounding the first compartment and being isolated from the external air, said second compartment being separated from the first compartment by a first partition.

For example, the first partition is porous in order to make possible the passage of the gas into the first compartment. The second compartment is pressurized by a gas, for example chosen from nitrogen, methane, argon, hydrogen, hydrogen sulfide and ammonia, or by heating.

For example, the device comprises an injector configured to inject a second gas into the second compartment of the first chamber and thus to pressurize said second compartment.

According to one embodiment, the second chamber comprises two separate compartments, the first compartment comprising a first opening, in fluidic communication with or connected to the second opening of the first chamber, and a second opening, on the side opposite the first opening, the second compartment at least partially surrounding the first compartment and being isolated from the external air, said second compartment being separated from the first compartment by a second partition and possessing a feed for the feeding of the second composition (B) into the second chamber.

For example, the device comprises an additional feed configured to inject a third gas into the second compartment of the second chamber and thus to pressurize said second compartment.

For example, the second partition is porous or perforated in order to make possible the passage of the second composition (B) into the first compartment of the second chamber. The second compartment is pressurized by a third gas (G3), for example chosen from nitrogen, methane, argon, hydrogen, hydrogen sulfide and ammonia, or by heating.

For example, the device further comprises a collecting system, for example coaxial with the two chambers, positioned above the second chamber and configured to stop the particles while allowing the gases to pass. In other words, the collecting system is permeable to gases. For example, the collecting system comprises a filtration system fitted inside said collecting system and a pressure-reducing system configured to create a negative pressure inside the collecting system.

The injection system, the first chamber, the second chamber and the collecting system are assembled, for example by screwing or welding, so as to ensure perfect leaktightness of the device making it possible to prevent access of external air to the inside of said device.

In a way which is not at all limiting, the injection system, the first chamber, the second chamber and the collecting system are positioned in an enclosure so as to ensure perfect leaktightness of the device making it possible to prevent access of external air to the inside of said enclosure. The interior of the enclosure is placed under negative pressure by the pressure-reducing system.

The invention also relates to a composition, in particular a cosmetic composition, comprising one or more particles of oxide of element M according to the invention.

### BRIEF DESCRIPTION OF THE FIGURES

A better understanding of the present invention will be obtained on studying the detailed description of embodiments, given by way of examples which are not at all limiting and illustrated by the appended drawings, not necessarily to scale, in which:

FIG. 1 represents a cross-sectional view of a particle of oxide of element M of formula (I) coated with a compound of formula (II) according to one embodiment of the invention;

FIG. 2 is a diagrammatic view of a flame spray pyrolysis device according to one embodiment of the invention; and

FIG. 3 is a diagrammatic view of a flame spray pyrolysis device according to another embodiment of the invention.

Other characteristics, aspects and advantages of the invention will become even more clearly apparent on reading the description and the example which follows.

In the present description, and unless otherwise indicated:
the expression “at least one” is equivalent to the expression “one or more” and can be replaced therewith;
the expression “of between” is equivalent to the expression “ranging from” and can be replaced therewith, and implies that the limits are included;
the expression “strictly between” is equivalent to the expression “strictly ranging from” and can be replaced therewith, and implies that the limits are not included;
the expression “strictly less than” implies that the superior limit is not included;
the expression “keratin materials” denotes in particular the skin and human keratin fibres, such as the hair;
the upper coating layer 2 is also referred to as “external layer”, “casing”, “coating” or “shell”;
the term “elements of column 3 of the Periodic Table of the Elements” is understood to mean, within the meaning of the present invention, scandium and yttrium. In other words, the elements of the family of the lanthanides and of the family of the actinides do not belong to the elements of column 3 of the Periodic Table of the Elements within the meaning of the invention;
the term “alkyl” is understood to mean an “alkyl radical”, that is to say a linear or branched C₁ to C₁₀, particularly C₁ to C₈, more particularly C₁ to C₆ and preferentially C₁ to C₄ hydrocarbon radical, such as methyl, ethyl, propyl, isopropyl, n-butyl, isobutyl or tert-butyl;
the term “aryl” radical is understood to mean a monocyclic or fused or non-fused polycyclic carbon-based group comprising from 6 to 22 carbon atoms, at least one ring of which is aromatic; preferentially, the aryl radical is a phenyl, biphenyl, naphthyl, indenyl, anthracenyl or tetrahydronaphthyl, preferably a phenyl;
the term “arylate” radical is understood to mean an aryl group which comprises one or more carboxylate —C(O)O″ groups, such as naphthalate or naphthenate;
the term “complexed metal” is understood to mean that the metal atom forms a “metal complex” or “coordination compound” in which the metal ion, corresponding to the central atom, i.e. M, is chemically bonded to one or more electron donors (ligands);
the term “ligand” is understood to mean a coordinating organic chemical group or compound, i.e. which comprises at least one carbon atom and which is capable of coordination with the metal M, and which, once coordinated or complexed, results in metal compounds corresponding to principles of a coordination sphere with a predetermined number of electrons (internal complexes or chelates)-see Ullmann's Encyclopedia of Industrial Chemistry, “Metal-Complex Dyes”, 2005, pp. 1-42. More particularly, the ligand(s) are organic groups which comprise at least one group which is electron-donating via an inductive and/or mesomeric effect, more particularly carrying at least one amino, phosphino, hydroxyl or thiol electron-donating group, or the ligand is a persistent carbene, particularly of “Arduengo” type (imidazol-2-ylidenes), or comprises at least one carbonyl group. Mention may more particularly be made, as ligand, of: i) those which contain at least one phosphorus-P<atom, i.e. phosphine, such as triphenylphosphines; ii) bidendate ligands of formula R—C(X)—CR′R″—C(X)—R′″ with R and R″, which are identical or different, representing a linear or branched (C₁-C₆)alkyl group and R′ and R″, which are identical or different, representing a hydrogen atom or a linear or branched (C₁-C₆)alkyl group, preferentially R′ and R″ representing a hydrogen atom, X representing an oxygen or sulfur atom or an N(R) group with R representing a hydrogen atom or a linear or branched (C₁-C₆)alkyl group, such as acetylacetone or β-diketones; iii) (poly) hydroxycarboxylic acid ligands of formula [HO—C(O)]ₙ-A-C(O)—OH and their deprotonated forms with A representing a monovalent group when n has the value zero or a polyvalent group when n is greater than or equal to 1, which is saturated or unsaturated, cyclic or non-cyclic and aromatic or non-aromatic based on a hydrocarbon comprising from 1 to 20 carbon atoms which is optionally interrupted by one or more heteroatoms and/or is optionally substituted, in particular by one or more hydroxyl groups; preferably, A represents a monovalent (C₁-C₆)alkyl group or a polyvalent (C₁-C₆)alkylene group optionally substituted by one or more hydroxyl groups; and n representing an integer of between 0 and 10 inclusive; preferably, n is between 0 and 5, such as among 0, 1 or 2; such as lactic, glycolic, tartaric, citric and maleic acids, and arylates, such as naphthalates; and iv) C₂ to C₁₀ polyol ligands comprising from 2 to 5 hydroxyl groups, in particular ethylene glycol or glycerol; more particularly still, the ligand(s) carry a carboxyl, carboxylate or amino group; particularly, the ligand is chosen from acetate, (C₁-C₆)alkoxylate, (di)(C₁-C₆)alkylamino and arylate, such as naphthalate or naphthenate, groups;
the term “combustible” is understood to mean a liquid compound or a gas which, with oxygen and energy, is consumed in a heat-generating chemical reaction: combustion. In particular the liquid combustibles are chosen from protic solvents, in particular alcohols, such as methanol, ethanol, ispropanol or n-butanol; aprotic solvents, in particular chosen from esters, such as methyl esters and those resulting from acetate, such as 2-ethylhexyl acetate, acids, such as 2-ethylhexanoic acid (EHA), acyclic ethers, such as ethyl ether, methyl tert-butyl ether (MTBE), methyl tert-amyl ether (MTAE), methyl tert-hexyl ether (MTHE), ethyl tert-butyl ether (ETBE), ethyl tert-amyl ether (ETAE) or diisopropyl ether (DIPE), cyclic ethers, such as tetrahydrofuran (THF), aromatic hydrocarbons or arenes, such as xylene, non-aromatic hydrocarbons; and their mixtures. The combustibles can optionally be chosen from liquefied hydrocarbons, such as acetylene, methane, propane or butane; and their mixtures.

### The Coated Particles of Oxide of Element M

The particle of oxide of element M, in particular of the type of oxide of M-M′ of core/shell structure, comprises:
(i) a core 1 constituted of at least one oxide of element M of formula (I):

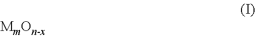

in which:
   M represents an element chosen from the alkali metals of column 1, the alkaline earth metals of column 2 and the elements of columns 3 to 14 of the Periodic Table of the Elements, bismuth, selenium, tellurium and astatine,
   m represents an integer greater than or equal to 1,
   n represents an integer greater than or equal to 1,
   x is equal to 0 or represents a non-integral number greater than 0 and strictly less than n, and
(ii) an upper coating layer 2, covering the surface of said core 1, constituted of at least one compound of formula (II):

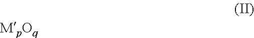

in which:
   M′ is an element, other than M, chosen from selenium and the elements of columns 4, 13 and 14 of the Periodic Table of the Elements,
   p represents an integer greater than or equal to 1,
   q represents an integer greater than or equal to 0.

Preferably, the element M represents an element chosen from the alkali metals of column 1, the alkaline earth metals of column 2 and the elements of columns 4 to 14 of the Periodic Table of the Elements, bismuth, selenium, tellurium and astatine.

Advantageously, the element M is other than gold, silver, iridium, platinum, palladium, rhodium, ruthenium, osmium and carbon.

More preferentially, the element M is chosen from iron, zinc, aluminium, silicon, selenium, sodium, potassium, magnesium and calcium.

More preferentially still, the element M is chosen from iron, zinc and aluminium.

Very particularly preferably, the element M is chosen from iron and zinc.

Preferably, the integer m ranges from 1 to 10, more preferentially from 1 to 5.

Preferably, the integer n ranges from 1 to 20, more preferentially from 2 to 10.

In particular, x is equal to 0 or represents a non-integral number strictly of less than n and of between 0 and 20.

According to a preferred embodiment of the invention, x is equal to 0.

According to another embodiment of the invention, x represents a non-integral number greater than 0 and strictly less than n; preferably strictly less than n and of between 0 and 20; more preferentially strictly less than n and of between 0 and 10.

Preferably, the oxide(s) of element M of formula (I) are chosen from: FeO, Fe₂O₃, Fe₃O₄, the compounds of formula FeO₁₋ₓ with x a non-integral number strictly of between 0 and 1, the compounds of formula Fe₃O₄₋ₓ with x a non-integral number strictly of between 0 and 4, the compounds of formula Fe₂O₃₋ₓ with x a non-integral number strictly of between 0 and 3, and the compounds of formula ZnO₁₋ₓ with x a non-integral number strictly of between 0 and 1.

More preferentially, the oxide(s) of element M of formula (I) are chosen from: FeO, Fe₃O₄, the compounds of formula FeO₁₋ₓ with x a non-integral number strictly of between 0 and 1, the compounds of formula Fe₃O₄₋ₓ with x a non-integral number strictly of between 0 and 4, the compounds of formula Fe₂O₃₋ₓ with x a non-integral number strictly of between 0 and 3, and the compounds of formula ZnO₁₋ₓ with x a non-integral number strictly of between 0 and 1.

According to the invention, the element(s) M in a particle are different from the element(s) M′ in this same particle.

Preferably, the core is in the crystalline state.

The crystalline state of the core 1 and also its composition can be determined, for example, by a conventional X-ray diffraction method.

Advantageously, the core 1 of the particle according to the invention is constituted of one or more aggregates of crystalline primary particles of oxide of element M. In other words, the core 1 is constituted of several microcrystals of oxide of element M.

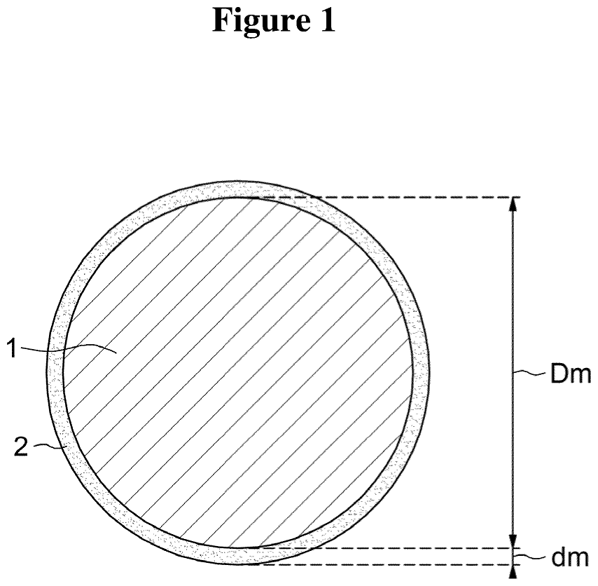

The coated particle of oxide of element M according to FIG. 1 comprises a core 1 of average diameter Dm, constituted of an oxide of element M of formula (I).

The coated particle of oxide of element M according to FIG. 1 also comprises an upper coating layer 2, constituted of a compound of formula (II), and completely covering the surface of the core 1 and of average thickness dm.

The number-average diameter Dm of the core 1 can, for example, be determined by transmission electron microscopy (abbreviated to TEM). Preferably, the number-average diameter Dm of the core 1 of the particle according to the invention is within the range extending from 3 to 5000 nm, more preferentially from 10 to 3000 nm and more preferentially still between 30 and 1000 nm.

The coated particle of oxide of element M according to the invention comprises an upper coating layer 2, covering the surface of the core 1, constituted of a compound of formula (II).

Advantageously, the upper coating layer 2 covers at least 90% of the surface of the core 1. More preferentially, the upper coating layer 2 covers the whole of the surface of the core 1.

The degree of coverage of the core by the upper coating layer can, for example, be determined by means of a visual analysis of TEM-BF or STEM-HAADF type, coupled to a STEM-EDX analysis.

Each of the analyses is carried out on a statistical number of particles, in particular on at least 20 particles. The particles are deposited on a metal grid made of a metal other than any metal forming part of the particles, whether in the core or in the upper coating layer. For example, the grid is made of copper (except in the case where it is desired to use copper in the manufacture of the particles).

Visual analysis of the TEM-BF and STEM-HAADF images makes it possible, based on the contrast, to deduce whether or not the coating completely surrounds the core of the particle. It is possible, by analysing each of the 20 (or more) images, to deduce therefrom a degree of coverage of the core and then, by taking the average, to determine an average degree of coverage.

The STEM-EDX analysis makes it possible to check that the coating indeed contains predominantly or exclusively the element M′. For this, it is necessary to make pointings (on at least 20 particles), on the edges of the particles. These pointings then reveal the element M′.

The STEM-EDX analysis also makes it possible to check that the core indeed contains the element M. For this, it is necessary to make pointings (on at least 20 particles), on the centres of the particles. These pointings then reveal the element M and the element M′.

According to the invention, the element(s) M′ are other than the element(s) M and chosen from selenium and the elements of columns 4, 13 and 14 of the Periodic Table of the Elements.

Preferably, the element M′ is chosen from selenium, titanium, aluminium and the elements of column 14 of the Periodic Table of the Elements.

More preferentially, the element M′ is chosen from selenium, titanium, aluminium, carbon and silicon.

Very particularly preferably, the element M′ is chosen from carbon and silicon.

According to a preferred embodiment, the element M′ is silicon.

According to another embodiment of the invention, the element M′ is carbon.

Preferably, the integer p ranges from 1 to 4. More preferentially, the integer p is equal to 1 or 2, and better still p is equal to 1.

Preferably, the integer q ranges from 0 to 4. More preferentially, the integer q is strictly greater than 0. More preferentially still, the integer q ranges from 1 to 4.

Preferably, the compound(s) of formula (II) are chosen from carbon, SiO₂, SnO₂ and Al₂O₃.

More preferentially, the compound(s) of formula (II) are chosen from carbon and SiO₂.

Advantageously, the element M exhibits a stoichiometric or non-stoichiometric intermediate oxidation number.

Within the meaning of the invention, the term “intermediate oxidation number” is understood to mean an oxidation number of between 0 (not included) and the maximum oxidation number of the element M (not included).

If the oxidation number is an integer, the expression “stoichiometric intermediate oxidation number” is then used. For example, if the element M exhibiting a stoichiometric intermediate oxidation number is iron, then the oxide(s) of element M of formula (I) can be chosen from FeO and Fe₃O₄. In another example, if the element M exhibiting a stoichiometric intermediate oxidation number is copper, then the oxide of element M of formula (I) can be Cu₂O.

If the oxidation number is non-integral, the expression “non-stoichiometric intermediate oxidation number” is then used. When the element M exhibits a non-stoichiometric intermediate oxidation number, the expression “suboxide of element M” is then used. For example, if the element M exhibiting a non-stoichiometric intermediate oxidation number is iron, then the suboxide(s) of element M of formula (I) can be chosen from the compounds of formula FeO₁₋ₓ, the compounds of formula Fe₃O₄₋ₓ and the compounds of formula Fe₂O₃₋ₓ. In another example, if the element M exhibiting a non-stoichiometric intermediate oxidation number is copper, then the suboxide of element M of formula (I) can be chosen from the compounds of formula CuO₁₋ₓ and the compounds of formula Cu₂O₁₋ₓ.

It has in particular been observed that the particles, the element(s) M of which exhibit an intermediate oxidation number, make it possible to obtain colourings of keratin materials which are even more intense and even more chromatic. In addition, these particles make it possible to obtain novel colourings.

Preferably, the oxide(s) of element M of formula (I) are chosen from the suboxides of element M of formula (I′):

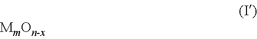

in which:
   M represents an element chosen from the alkali metals of column 1, the alkaline earth metals of column 2 and the elements of columns 3 to 14 of the Periodic Table of the Elements, bismuth, selenium, tellurium and astatine,
   m represents an integer greater than or equal to 1,
   n represents an integer greater than or equal to 1,
   x represents a non-integral number greater than 0 and strictly less than n.

The preferences and the characteristics of the element M and of the numbers m and n of the formula (I) which is described above also apply to the element M and to the numbers m and n of the formula (I′).

Preferably, x represents a non-integral number strictly of less than n and of between 0 and 20; more preferentially strictly of less than n and of between 0 and 10.

Preferably, the suboxide(s) of element M of formula (I′) are chosen from the compounds of formula FeO₁₋ₓ with x a non-integral number strictly of between 0 and 1, the compounds of formula Fe₃O₄₋ₓ with x a non-integral number strictly of between 0 and 4, the compounds of formula Fe₂O₃₋ₓ with x a non-integral number strictly of between 0 and 3, and the compounds of formula ZnO₁₋ₓ with x a non-integral number strictly of between 0 and 1.

According to a preferred embodiment of the invention, the particles comprise:
a core 1 constituted of an oxide of element M of formula (I) or (I′) chosen from FeO, Fe₂O₃, Fe₃O₄, the compounds of formula FeO₁₋ₓ with x a non-integral number strictly of between 0 and 1, the compounds of formula Fe₃O₄₋ₓ with x a non-integral number strictly of between 0 and 4, the compounds of formula Fe₂O₃₋ₓ with x a non-integral number strictly of between 0 and 3, and the compounds of formula ZnO₁₋ₓ with x a non-integral number strictly of between 0 and 1; and
an upper coating layer 2, covering the surface of said core 1, constituted of a compound of formula (II) chosen from carbon, SiO₂, SnO₂ and Al₂O₃.

According to another particularly preferred embodiment of the invention, the particles comprise:
a core 1 constituted of an oxide of element M of formula (I) or (I′) chosen from FeO, Fe₂O₃, Fe₃O₄, the compounds of formula FeO₁₋ₓ with x a non-integral number strictly of between 0 and 1, the compounds of formula Fe₃O₄₋ₓ with x a non-integral number strictly of between 0 and 4, the compounds of formula Fe₂O₃₋ₓ with x a non-integral number strictly of between 0 and 3, and the compounds of formula ZnO₁₋ₓ with x a non-integral number strictly of between 0 and 1; and
an upper coating layer 2, covering the surface of said core 1, constituted of a compound of formula (II) chosen from carbon and SiO₂.

The number-average thickness dm of the upper coating layer can also be determined by transmission electron microscopy.

Preferably, the number-average thickness dm of the upper coating layer is within the range extending from 1 to 20 nm, more preferentially from 1 to 10 nm and more preferentially still from 2 to 6 nm.

Advantageously, the upper coating layer 2 is amorphous.

Advantageously, the upper coating layer 2 is transparent.

Advantageously, the particle according to the invention comprises element M and element M′ according to a specific (M/M′) molar atomic ratio.

This ratio corresponds to the amount in moles of element M present in the particle according to the invention, on the one hand, to the amount in moles of element M′ present in the particle according to the invention, on the other hand.

This ratio can be determined by spectrometry according to one of the following two methods. According to a first method, powder is spread out and an X-ray fluorimetry study is carried out with an X-ray spectrometer to deduce therefrom the metal ratio. According to another method, the particles of the invention are dissolved beforehand in an acid. Then an elemental analysis is carried out on the material obtained by ICP-MS (Inductively Coupled Plasma Mass Spectrometry) to deduce therefrom the metal ratio.

Preferably, the (M/M′) molar atomic ratio of the particle according to the invention is strictly greater than 0.3; more preferentially greater than or equal to 1; more preferentially still greater than or equal to 3; better still within the range extending from 3 to 100; and even better still within the range extending from 3 to 10.

The number-average diameter of the particle according to the invention may also be determined by transmission electron microscopy. Preferably, the number-average diameter of the particle according to the invention is within the range extending from 4 to 5000 nm; more preferentially from 10 to 3000 nm; and more preferentially still from 30 to 1000 nm.

Preferably, the BET specific surface of the particle according to the invention is between 1 m²/g and 200 m²/g, more preferentially between 30 and 100 m²/g.

According to a specific embodiment of the invention, the coated particle according to the invention can optionally further comprise an additional coating layer covering the upper coating layer 2 and comprising at least one hydrophobic organic compound.

The hydrophobic organic compound(s) included in the additional coating layer are more preferentially chosen from silicones, in particular silicones comprising at least one fatty chain; carbon-based derivatives comprising at least 6 carbon atoms, in particular fatty acid esters; and their mixtures.

The additional coating layer can be produced by a liquid route or by a solid route. By a liquid route, the hydroxyl functions are reacted with reactive functions of the compound which will form the coating (typically silanol functions of a silicone or the acid functions of a carbon-based fatty substance). By a solid route, the particles are brought into contact with a liquid or pasty compound comprising the hydrophobic substance.

Preferably, the coated particles according to the invention are obtained by the preparation process of the invention as described below.

### The Process for the Preparation of the Coated Particles

Another subject-matter of the invention relates to a process for the preparation of particles of oxide of element M of formula (I) which are coated with a compound of formula (II), in particular of the type of oxide of M-M′ of core/shell structure, comprising at least one stage a. of preparation of a composition (A), then a stage b. of formation of the flame and a stage c. of injection of a composition (B).

Stage a. of the process according to the invention consists of the preparation of a composition (A) by adding one or more precursors of element M to a combustible solvent or to a mixture of combustible solvents.

According to the invention, the element M represents an element chosen from the alkali metals of column 1, the alkaline earth metals of column 2 and the elements of columns 3 to 14 of the Periodic Table of the Elements, bismuth, selenium, tellurium and astatine.

Preferably, the element M represents an element chosen from the alkali metals of column 1, the alkaline earth metals of column 2 and the elements of columns 4 to 14 of the Periodic Table of the Elements, bismuth, selenium, tellurium and astatine.

Advantageously, the element M is other than gold, silver, iridium, platinum, palladium, rhodium, ruthenium, osmium and carbon.

More preferentially, the element M is chosen from iron, zinc, aluminium, silicon, selenium, sodium, potassium, magnesium and calcium.

More preferentially still, the element M is chosen from iron, zinc and aluminium.

Very particularly preferably, the element M is chosen from iron and zinc. According to the invention, the element(s) M are other than the elements M′.

The precursors of the element M and the combustible solvents which can be used according to the invention can be chosen from the precursors of the element M and the combustible solvents conventionally used in flame spray pyrolysis.

Preferably, the precursors of the element M included in the composition (A) are chosen from:
the nitrates of element M, for example iron nitrate,
the sulfates of element M, for example iron sulfate,
the compounds comprising one or more elements M complexed or not to one or more ligands containing at least one carbon atom, such as, for example, carbonates, acetates and citrates,
and their mixtures.

More preferentially, said ligand(s) are chosen from acetate, (C₁-C₆)alkoxylate, (C₂-C₁₀)alkylcarboxylate, (di)(C₁-C₆)alkylamino, and arylate, such as naphthalate or naphthenate, groups.

More preferentially, the precursors of element M included in the composition (A) are chosen from the nitrates of elements M.

Preferably, the combustible solvent(s) are chosen from protic combustible solvents, aprotic combustible solvents, and their mixtures; more preferentially from alcohols, esters, acids, acyclic ethers, cyclic ethers, aromatic hydrocarbons or arenes, non-aromatic hydrocarbons, such as liquefied hydrocarbons, for example acetylene, methane, propane or butane, and their mixtures; and better still from 2-ethylhexyl acetate, 2-ethylhexanoic acid (EHA), ethyl ether, methyl tert-butyl ether (MTBE), methyl tert-amyl ether (MTAE), methyl tert-hexyl ether (MTHE), ethyl tert-butyl ether (ETBE), ethyl tert-amyl ether (ETAE), diisopropyl ether (DIPE), tetrahydrofuran (THF), xylene and their mixtures.

In particular, the combustible solvent(s) can be chosen from aprotic combustible solvents comprising at least three carbon atoms and their mixtures; and better still from xylene, toluene, tetrahydrofuran, 2-ethylhexyl acetate, 2-ethylhexanoic acid (EHA) and their mixtures.

According to a specific embodiment of the invention, the composition (A) comprises a mixture of combustible solvents, preferably comprising at least two of the following combustible solvents: 2-ethylhexanoic acid (EHA), toluene, absolute ethanol and diethylene glycol monobutyl ether.

Better still, the composition (A) comprises a mixture of combustible solvents constituted of 2-ethylhexanoic acid (EHA), toluene, absolute ethanol and diethylene glycol monobutyl ether.

Better still, the composition (A) comprises a mixture of combustible solvents constituted of at least 5% by volume of 2-ethylhexanoic acid (EHA), of at least 5% by volume of toluene, of at least 5% by volume of absolute ethanol and of at least 5% by volume of diethylene glycol monobutyl ether, with respect to the total volume of the mixture of combustible solvents.

Advantageously, the content of precursor of the element M in the composition (A) is between 1% and 60% by weight, preferably between 15% and 30% by weight, with respect to the total weight of the composition (A).

The preparation process according to the invention further comprises a stage b. of injection of the composition (A) and of an oxygen-containing gas (G) into a flame spray pyrolysis (FSP) device 10 in order to form a flame.

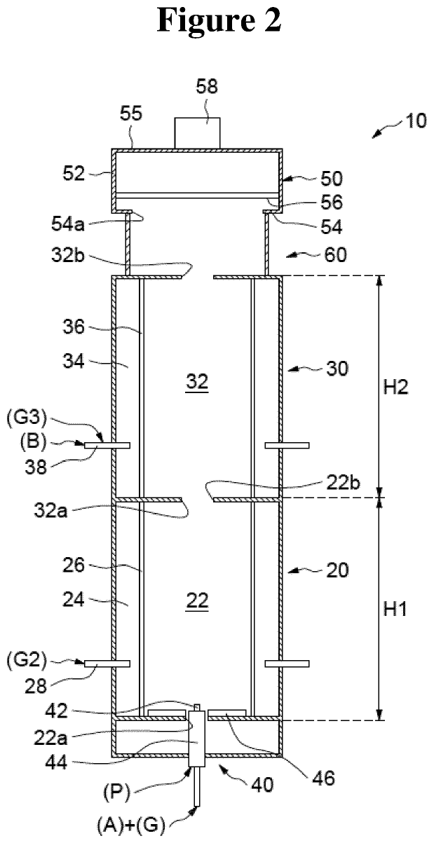

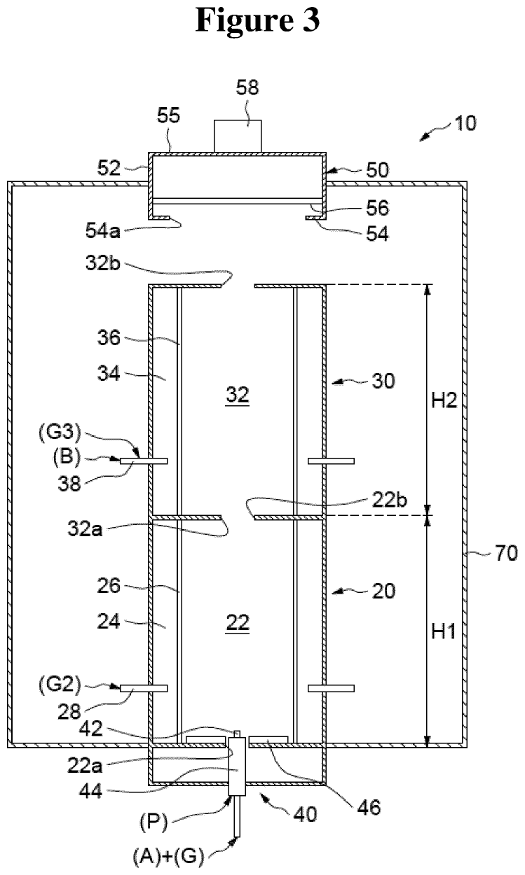

The flame spray pyrolysis device 10 will be described more specifically below with reference to FIGS. 2 and 3.

During this stage b., the composition (A) and the oxygen-comprising gas (G) are advantageously injected into the flame spray pyrolysis device 10.

Preferably, the flame formed during stage b. is at a temperature of greater than or equal to 2000° C., at at least one point of the flame.

Stage b. can optionally further comprise an additional injection of a “premix” mixture (P) comprising oxygen and one or more combustible gases, such as methane. This “premix” mixture (also referred to as “supporting flame oxygen”) makes possible the production of a support flame intended to ignite and maintain the flame resulting from the composition (A) and the oxygen-comprising gas (G) (i.e. “dispersion oxygen”). The mixture of the composition (A) with the gas (G), on the one hand, and the premix (P), on the other hand, are injected separately, that is to say that the mixture of the composition (A) with the oxygen-comprising gas (G) is injected by means of one tube and that the premix (P) is injected by means of another tube.

Preferably, during stage b., the composition (A), the oxygen-comprising gas (G) and optionally the “premix” mixture (P), when it is present, are injected into a reaction tube (also referred to as “enclosing tube”). Preferably, this reaction tube is made of metal or of quartz. Advantageously, the reaction tube exhibits a height of greater than or equal to 30 cm, preferably of greater than or equal to 40 cm and more preferentially of greater than or equal to 50 cm. Preferentially, the length of said reaction tube is between 30 cm and 300 cm, particularly between 40 cm and 200 cm and more particularly between 45 cm and 100 cm, such as 50 cm.

The ratio by weight of the weight of solvent(s) present in the composition (A), on the one hand, to the weight of oxygen-containing gas (G), on the other hand, is defined as follows:

Firstly, the amount of oxygen-containing gas (also referred to as “oxidizer compound”) for the assembly formed by the composition (A), that is to say the combustible solvent(s) and the precursor(s) of the element M, on the one hand, and the oxygen-containing gas, on the other hand, to be able to react together in a combustion reaction in a stoichiometric ratio (thus without an excess or deficit of oxidizer compound) is calculated.

Starting from this calculated amount of oxygen-containing gas (also referred to as “calculated oxidizer”), a new calculation is performed to deduce therefrom the amount of oxygen-containing gas to be injected (also referred to as “oxidizer to be injected”), according to the formula:

### Oxidizer to be injected=Calculated oxidizer/φ

with φ a correction factor, preferably of between 1 and 2.5, more preferentially between 1.2 and 2, more preferentially still between 1.3 and 1.8, better still between 1.4 and 1.6.

This method is in particular defined by Turns, S. R. in An Introduction to Combustion: Concepts and Applications, 3rd ed.; McGraw-Hill: New York, 2012.

Preferably, the molar amount of oxygen-containing gas (G) to be injected during stage b. is strictly less than the molar amount of oxygen-containing gas necessary to cause the composition (A) to react with the oxygen in a stoichiometric ratio.

The flame spray pyrolysis device 10 which can be used in the preparation process according to the invention can comprise one or more chambers. Preferably, the flame spray pyrolysis device 10 which can be used in the preparation process according to the invention comprises several chambers, more preferentially two chambers.

Preferably, said flame spray pyrolysis device 10 is pressurized by an inert gas (G2) chosen, for example, from nitrogen, methane, argon, hydrogen, hydrogen sulfide and ammonia; more preferentially from nitrogen, methane, hydrogen and argon; more preferentially still from nitrogen and argon, and better still by nitrogen.

According to a preferred embodiment of the invention, when the flame spray pyrolysis device 10 comprises only a single chamber, the chamber of said flame spray pyrolysis device 10 is pressurized by an inert gas (G2) chosen, for example, from nitrogen, methane, argon, hydrogen, hydrogen sulfide and ammonia; preferably from nitrogen, methane, hydrogen and argon; more preferentially from nitrogen and argon, and better still by nitrogen.

According to another preferred embodiment of the invention, when the flame spray pyrolysis device 10 comprises several chambers, the first chamber 20 of said flame spray pyrolysis device 10 is pressurized by an inert gas (G2) chosen, for example, from nitrogen, methane, argon, hydrogen, hydrogen sulfide and ammonia; preferably from nitrogen, methane, hydrogen and argon; more preferentially from nitrogen and argon, and better still by nitrogen.

Preferably, the flow rate of inert gas (G2)_{injected} into the flame spray pyrolysis device 10 ranges from 5 l/min to 70 1/min; more preferentially from 10 1/min to 50 1/min.

More preferentially, the flow rate of nitrogen (G2)_{injected} into the flame spray pyrolysis device 10 ranges from 5 l/min to 70 1/min; more preferentially from 10 1/min to 50 1/min.

According to a particularly preferred embodiment of the invention, the correction factor q is between 1 and 2.5, more preferentially between 1.2 and 2, more preferentially still between 1.3 and 1.8, better still between 1.4 and 1.6; and the flow rate of inert gas (G2), more particularly nitrogen, injected into the flame spray pyrolysis device 10 ranges from 5 l/min to 70 1/min; more preferentially from 10 1/min to 50 1/min.

The preparation process according to the invention further comprises a stage c. comprising the injection of a composition (B) comprising one or more precursors of element M′ until an upper coating layer 2 constituted of element M′ or of oxide(s) of element M′ is obtained, at the surface of said aggregates of oxide of element M.

As indicated above, according to the invention, the element(s) M′ are other than the element(s) M and chosen from selenium and the elements of columns 4, 13 and 14 of the Periodic Table of the Elements.

Preferably, the element M′ is chosen from selenium, titanium, aluminium and the elements of column 14 of the Periodic Table of the Elements.

More preferentially, the element M′ is chosen from selenium, titanium, aluminium, carbon and silicon.

Very particularly preferably, the element M′ is chosen from carbon and silicon.

According to a preferred embodiment, the element M′ is silicon.

According to another embodiment of the invention, the element M′ is carbon. Preferably, the precursor of element M′ comprises at least two M′ atoms and several M′-carbon covalent bonds. More preferentially, the precursor of element M′ comprises at least three M′ atoms and several M′-carbon covalent bonds.

More preferentially, the precursor of element M′ is chosen from hexa (di)(C₁-C₄)alkyldisiloxanes, such as hexadimethyldisiloxane, (di)(tri)(tetra)(C₁-C₄)alkoxysilanes, such as tetraethoxysilane, bis[(di)(tri)alkoxysilyl](C₁-C₄)alkanes, such as 1,2-bis(triethoxysilyl)ethane or 1,2-bis(trimethoxysilyl)ethane, (C₁-C₄)alkoxy (di)(tri)(C₁-C₄)alkylsilanes, such as methoxytrimethylsilane, hydrocarbon gases, such as acetylene, aluminium (di)(C₁-C₆)alkoxylates, aluminium (di)(C₁-C₆)alkylcarboxylates, such as aluminium diacetate hydroxide, (poly) (C₁-C₆)alkoxylate stannates, (poly) (C₁-C₆)alkylcarboxylate stannates, such as tetraacetate stannate, and their mixtures.

More preferentially still, the precursor of element M′ is chosen from hexadimethyldisiloxane, tetraethoxysilane, 1,2-bis(triethoxysilyl)ethane, 1,2-bis(trimethoxysilyl)ethane, methoxytrimethylsilane and their mixtures.

According to a specific embodiment of the invention, the composition (B) can be injected with an inert gas (G3) chosen, for example, from nitrogen, methane, argon, hydrogen, hydrogen sulfide and ammonia; preferably from nitrogen, methane, hydrogen and argon; and more preferentially from nitrogen and argon.

For example, nitrogen can be bubbled into the composition (B), prior to its injection during stage c. The flow rate of injection of the composition (B) can subsequently be controlled by determination of the known pressure by a person skilled in the art, such as, for example, the method defined by Scott, D. W.; Messerly, J. F.; Todd, S. S.; Guthrie, G. B.; Hossenlopp, I. A.; Moore, R. T.; Osborn, A. Berg, W. T.; Mccullough, J. P., Hexamethyldisiloxane: Chemical Thermodynamic Properties and Internal Rotation about the Siloxane Linkage, J. Phys. Chem., 1961, 65, 1320-6.

Preferably, the content of precursor(s) of element M′ in the composition (B) injected during stage c. of the process according to the invention is between 1% and 60% by volume, more preferentially between 5% and 30% by volume, with respect to the total volume of the composition (B).

Advantageously, the composition (B) can further comprise one or more solvents. Preferably, the solvent(s) present in the composition (B) are chosen from polar protic solvents other than water; and more preferentially from (C₁-C₈)alkanols. More preferentially still, the composition (B) comprises ethanol.

Preferably, the solvent(s) present in the composition (B) are chosen from solvents which are combustible at the flame temperature of stage c., preferably combustible at a temperature of between 200° C. and 600° C. and more preferentially between 300° C. and 400° C. Better still, the solvent(s) present in the composition (B) have a boiling point of greater than or equal to ambient temperature (25° C.) and more preferentially of between 50° C. and 120° C.

During the process according to the invention, an (M/M′)_{injected} molar atomic ratio can be calculated. This ratio corresponds to the amount in moles of atoms of element M injected during stage b., on the one hand, to the amount in moles of element M′ injected during stage c., on the other hand.

Preferably, the (M/M′)_{injected} molar atomic ratio is greater than or equal to 0.25; more preferentially included in the range extending from 0.25 to 120; more preferentially still from 0.25 to 99; better still included in the range extending from 1 to 80; and even better still included in the range extending from 3 to 20.

According to the invention, the flame spray pyrolysis device 10 is isolated from the external air, so that the amount of oxygen present in said device 10 is controlled, and more preferentially so that the oxygen present in said device 10 originates solely from said gas (G) and optionally from the mixture (P). In other words, the oxygen of the air cannot enter the combustion chamber(s) and react with the composition (A) and the solvent(s).

Preferably, stage b. is carried out in a first chamber of the flame spray pyrolysis device 10 and stage c. is carried out in a second chamber of said device 10. As illustrated in FIGS. 2 and 3, said second chamber 30 is contiguous with the first chamber 20 and extends said first chamber. In an alternative form, provision might be made for the two chambers to be connected by a pipe.

The invention also relates to the coated particles of oxide of element M of formula (I) or (I′) obtained according to the preparation process according to the invention described above.

### The Flame Spray Pyrolysis Device

An example of a flame spray pyrolysis device 10 is illustrated in FIG. 2. The flame spray pyrolysis device 10 comprises a first chamber 20 employing the composition (A) and the oxygen-containing gas (G), and a second chamber 30 employing the composition (B) comprising one or more precursors of element M′.

The flame spray pyrolysis device 10 further comprises an injection system 40 comprising a first tube 42 emerging in the first chamber 20 and delivering the composition (A) and the oxygen-containing gas (G), and a second tube 44 emerging in the first chamber 20 and delivering the “premix” mixture (P) comprising oxygen and one or more combustible gases, such as methane. The second tube 44 makes it possible to ensure a flame necessary for the ignition of the compounds resulting from the first tube 42.

The first and second tubes 42, 44 are separate from one another.

As illustrated, the first and second tubes 42, 44 are coaxial and the second tube 44 at least partially surrounds the first tube 42.

In a way which is not at all limiting, the injection system 40 of the device 10 further comprises an additional feed 46 in the first chamber 20 of an inert gas, such as, for example, nitrogen. The additional feed 46 can be provided in the form of a porous part, from where the inert gas can emerge under pressure between 2 and 20 bar (i.e. between 2×10⁵ and 20×10⁵ Pa).

The composition (A), the oxygen-containing gas (G) and the combustible (P) which result from the injection system 40 are incinerated in the first chamber 20.

As illustrated, the first chamber 20 comprises two separate compartments 22, 24. The first compartment 22 comprises a first lower opening 22a in which the injection system 40 emerges and a second upper opening 22b, on the side opposite the first opening 22a.

The second compartment 24 surrounds the first compartment 22 and is isolated from the external air. The second compartment 24 is separated from the first compartment 22 by a gas-permeable partition 26.

The second compartment 24 comprises an upper wall, a lower wall and side walls (not referenced) forming a closed housing isolated from the external air.

The second compartment 24 is pressurized by a gas (G2), for example chosen from nitrogen, methane, argon, hydrogen, hydrogen sulfide and ammonia. The gas (G2) is injected into the second compartment 24 via an injector 28. For example, the injector 28 comprises a single tube emerging in the second compartment 24. In an alternative form, the injector 28 can comprise two or more tubes emerging in the second compartment 24. The tubes may or may not be evenly spaced over the circumference of the second compartment 24.

The partition 26 for separation of the two compartments 22, 24 is configured in order to make possible the passage of the gas (G2) into the first compartment 22. For example, the partition 26 is made of porous material. The porosity of the partition 26 is, for example, between 10 μm and 100 μm.

The first chamber 20 exhibits a height H1, for example of between 10 cm and 1 m.

The second chamber 30 is configured in order to employ the composition (B) comprising one or more precursors of element M′.

As illustrated, the second chamber 30 comprises two separate compartments 32, 34. The first compartment 32 comprises a first lower opening 32a coinciding with the second opening 22b of the first chamber 20, and a second upper opening 32b, on the side opposite the first opening 32a.

In an alternative form, the first lower opening 32a might be offset laterally from the second opening 22b of the first chamber 20. Provision might also be made for the first chamber 20 to be connected to the second chamber 30 by a pipe.

The second compartment 34 surrounds the first compartment 32 and is isolated from the external air. The second compartment 34 is separated from the first compartment 32 by a gas-permeable partition 36.

The second compartment 34 comprises an upper wall, a lower wall and side walls (not referenced) forming a closed housing isolated from the external air.

The second compartment 34 has a feed 38 for feeding the composition (B) into the second chamber 30.

The feed 38 is pressurized by a gas (G3), for example chosen from nitrogen, methane, argon or hydrogen, or by the heating of the composition (B). For example, the feed 38 comprises a single tube emerging in the second compartment 34. In an alternative form, the feed 38 comprises two or more tubes emerging in the second compartment 34. The tubes may or may not be evenly spaced over the circumference of the second compartment 34.

The partition 36 for separation of the two compartments 32, 34 is configured in order to make possible the passage of the composition (B) from the second compartment 34 to the first compartment 32. For example, the partition 36 comprises a plurality of perforations (not represented), from approximately 0.1 mm to 0.5 mm, in number from 1 to 10 perforations per cm² of the separating partition 36.

The second chamber 30 exhibits a height H2, for example of between 10 cm and 1 m.

Preferably, the height H1 of the first chamber 20 is equal to the height H2 of the second chamber 30, plus or minus 10%. Preferably, the dimensions of the first chamber 20 are equal to the dimensions of the second chamber 30.

The flame spray pyrolysis device 10 further comprises a collecting system 50 configured to stop the particles while allowing the gases to pass.

The collecting system 50 is in this instance coaxial with the two chambers 20, 30 and positioned above the second chamber 30. In an alternative form, provision might be made for the collecting system 50 to be offset laterally from the chambers 20, 30.

The collecting system 50 is delimited radially by one or more side partitions 52 and axially by a lower wall 54 comprising an opening 54a emerging in the second chamber 30 and an upper wall 55 on the side opposite the lower wall 54.

The collecting system 50 further comprises a filtration system 56 fitted inside said collecting system between the side walls 52, and a pressure-reducing system 58, such as, for example, a pump, fitted to the upper wall 55 of said system 50.

The pump 58 is configured in order to create a negative pressure inside the collecting system 50 in order to isolate the chambers 20, 30 from the external air. Advantageously, the negative pressure inside the collecting system 50 is of the order of 0.5 to 0.8 bar (i.e., between 5×10⁴ and 8×10⁴ Pa).

In a way which is not at all limiting, the collecting system 50 is spaced out axially from the second chamber 30 by a spacer 60.

As illustrated in FIG. 2, the injection system 40, the first chamber 20, the second chamber 30 and the collecting system 50, indeed even the spacer 60 when it is present, are assembled, for example by screwing or welding, so as to ensure perfect leaktightness of the device 10, and in particular of the chambers 20, 30, making it possible to prevent the access of external air to the inside of said device 10.

The embodiment illustrated in FIG. 3, in which the same elements carry the same references, differs from the embodiment illustrated in FIG. 2 only in that the injection system 40, the first chamber 20, the second chamber 30 and the collecting system 50, indeed even the spacer 60 when it is present, are positioned in an enclosure 70, so as to ensure perfect leaktightness of the device 10, and in particular of the chambers 20, 30, making it possible to prevent the access of external air to the inside of said enclosure 70. The interior of the enclosure 70 is placed under negative pressure by the pump 58.

According to a preferred embodiment of the device, said device exhibits an axis of symmetry Δ which passes through the centre/middle of the injection system 40 and through the centre/middle of the collecting system 50. More preferentially, the device is symmetrical and in particular cylindrical, passing through said axis of symmetry Δ.

### The Composition

Another subject-matter of the invention relates to a composition, preferably a cosmetic composition, comprising one or more coated particles of oxide of element M of formula (I) or (I′) as described above, and/or preferably obtained by the preparation process according to the invention.

The composition according to the invention is intended to be applied to keratin materials, preferably the skin (in particular the face) and/or the hair, in order to dye and/or make up the keratin materials. An optional stage of drying the keratin materials can be carried out.

The composition according to the invention can be in various presentation forms. Thus, the composition according to the invention can be in the form of a powdered (pulverulent) composition or of a liquid composition, or in the form of a milk, of a cream, of a paste or of an aerosol composition.

The compositions according to the invention are in particular cosmetic compositions, i.e. the material(s) of the invention are in a cosmetically acceptable carrier. The term “cosmetically acceptable carrier” is understood to mean a medium which is appropriate for application to keratin materials, in particular human keratin materials, such as the skin, said cosmetically acceptable carrier being generally constituted of water or of a mixture of water and of one or more organic solvents or of a mixture of organic solvents.

The composition according to the invention is advantageously an aqueous composition.

Preferably, the composition comprises water in a content in particular of between 5% and 95% inclusive, with respect to the total weight of the composition.

Within the meaning of the invention, the term “organic solvent” is understood to mean an organic substance capable of dissolving another substance without chemically modifying it.

Mention may be made, by way of organic solvent, for example, of lower C₂-C₆ alkanols, such as ethanol and isopropanol; polyols and polyol ethers, such as 2-butoxyethanol, propylene glycol, propylene glycol monomethyl ether and diethylene glycol monoethyl ether and monomethyl ether, and also aromatic alcohols, such as benzyl alcohol or phenoxyethanol, and their mixtures.

Preferably, the organic solvents are present in the composition according to the invention in a content of inclusively between 0.1% and 40% by weight approximately, with respect to the total weight of the composition, and more preferentially between 1% and 30% by weight approximately and more particularly still of inclusively between 5% and 25% by weight, with respect to the total weight of the composition.

The compositions of the invention can include a fatty phase and be in the form of direct or inverse emulsions.

The composition according to the invention can be prepared according to the techniques well known to a person skilled in the art, in the form of a simple or complex emulsion (oil-in-water, or abbreviated to O/W, water-in-oil or W/O, oil-in-water-in-oil or O/W/O, or water-in-oil-in-water or W/O/W), such as a cream, a milk or a cream gel.

According to a specific embodiment of the invention, the composition according to the invention can also be provided in the form of an anhydrous composition, such as, for example, in the form of an oil. The term “anhydrous composition” is understood to mean a composition containing less than 2% by weight of water, preferably less than 1% by weight of water and more preferentially still less than 0.5% by weight of water, with respect to the total weight of the composition, and indeed even a composition devoid of water. In compositions of this type, the water possibly present is not added during the preparation of the composition but corresponds to the residual water contributed by the mixed ingredients.

The particle(s) according to the invention can also be in dry form (powder, flakes, plates), as a dispersion or as a liquid suspension or as an aerosol. The particle(s) according to the invention can be used as is or mixed with other ingredients.

Preferably, the compositions of the invention contain between 0.1% and 40% by weight of particles according to the invention, more preferentially between 0.5% and 20% by weight, more preferentially still between 1% and 10% by weight and better still between 1.5% and 5% by weight, with respect to the total weight of the composition.

The compositions of the invention can be used in single application or in multiple application. When the compositions of the invention are intended for a multiple application, the content of particles of the invention is generally lower than in the compositions intended for a single application.

Within the meaning of the present invention, the term “single application” is understood to mean just one application of the composition, it being possible for this application to be repeated several times per day, each application being separated from the next by one or more hours, or an application once each day, depending on the need.

Within the meaning of the present invention, the term “multiple application” is understood to mean an application of the composition repeated several times, in general from 2 to 5 times, each application being separated from the next by a few seconds to a few minutes. Each multiple application can be repeated several times per day, separated from the next by one or more hours, or each day, depending on the need.

The following examples serve to illustrate the invention without, however, exhibiting a limiting nature.

### EXAMPLES

### Example 1

1.1 In a first step, a composition (A) was prepared from manganese nitrate (200 mM) in a mixture of combustible solvents: acetonitrile and ethyl hexanoate.

Non-coated manganese oxide MnO particles P1 were subsequently prepared using a conventional FSP preparation process Prep 1 with the pre-prepared composition (A) (outside the invention).

The parameters of the Prep 1 process are as follows:
ratio (composition (A)/O₂)=5 ml/min of composition (A) and 5 l/min of gas (O₂),
a gas mixture for providing the flame of 1 1/min of methane and 2 1/min of oxygen,
a stream of inert gas (G2) of 10 1/min of nitrogen is injected into the FSP device.

1.2 Particles of manganese oxide MnO coated with silicon dioxide P2 were subsequently prepared in a double-chamber FSP device using the preparation process Prep 2 according to the invention with a composition (A) comprising manganese nitrate (200 mM) in a mixture of combustible solvents (acetonitrile+ethyl hexanoate) and a gas composition (B) comprising hexadimethyldisiloxane and nitrogen, in a proportion such that the Mn/Si_{particle} molar atomic ratio=0.49.

The parameters of the Prep 2 process are as follows:
the composition (A) and the oxygen-containing gas (G) are injected into the 1st chamber of the FSP device, according to a (composition (A)/O₂) ratio=5 ml/min of composition (A) and 5 l/min of gas (O₂),
a gas mixture for providing the flame of 1 l/min of methane and 2 1/min of oxygen,
a stream of inert gas (G2) of 10 1/min of nitrogen is injected into the 1st chamber of the double-chamber FSP device,
the composition (B) is injected into the second chamber of the FSP device by means of a 5 l/min nitrogen stream.

The factor φ for regulating the oxygen flow rate is identical in the preparation process Prep 1 and in the preparation process Prep 2.

1.3 It was observed that the manganese oxide particles obtained were crystalline.

It was also observed that the non-coated manganese oxide particles P1 (outside the invention) have a green-grey colour. The particles P1 according to the process Prep 1 have a number-average diameter equal to 50 nm.

Furthermore, the particles P2 obtained according to the process Prep 2 according to the invention have an intense green colour, are coated with an upper layer of silicon dioxide with a thickness of approximately 20 nm and exhibit a (Mn/Si) particle atomic ratio of 0.49.

The particles P2 according to the process Prep 2 have a number-average diameter equal to 70 nm.

1.4 The colorimetric data of the particles P1 and P2 were measured in the CIELab system with a Data Color SF600X spectrophotometer (illuminant D65, angle 10° and specular component included). In this L*a* b* system, L*represents the lightness, a*indicates the green/red colour axis and b* indicates the blue/yellow colour axis. The higher the value of L, the lighter or less intense the colour. Conversely, the lower the value of L*, the darker or more intense the colour. The higher the value of a*, the redder the shade, and the higher the value of b*, the yellower the shade.

The results are collated in the table 1 below:

TABLE 1

| | L* | a* | b* |
|---|---|---|---|
| Particles P1 according to the process | 58 | 7 | 15 |
| Prep 1 (outside the invention) | | | |
| Particles P2 according to the process | 54 | 19 | 21 |
| Prep 2 (invention) | | | |

It was observed that the green colour of the coated manganese oxide particles P2 according to the process Prep 2 (invention) is more intense than the green-grey colour of the non-coated manganese oxide particles P1 according to the process Prep 1 (outside the invention).

It was also observed, after storage in the open air and at ambient temperature and ambient external light for 3 months, that the colour of the coated manganese oxide particles P2 according to the process Prep 2 (invention) did not vary over time, unlike the colour of the non-coated manganese oxide particles P1 according to the process Prep 1 (outside the invention), which changed over time towards dark grey.

### Example 2

A composition C₁ (invention), comprising 0.45 g of manganese oxide particles P2 coated with silicon dioxide according to the above process Prep 2 and 9 ml of isododecane, was prepared.

A composition C2 (outside the invention), comprising 0.45 g of commercial pigment PG-7 (based on copper phthalocyanine), 0.25 g of commercial pigment PY-42 and 9 ml of isododecane, was prepared. The colour of the composition C₁ is similar to that of the composition C₂.

The following protocol is subsequently repeated ten times for each of the compositions C₁ and C₂:
stirring of the composition for 15 seconds, then
application of 0.4 g of composition to a surface equivalent to the skin, then
drying in the open air.

Each of the 10 coatings thus prepared per composition was subsequently compared visually.

It was observed that the green colour is identical for each of the 10 coatings obtained with the composition C₁ according to the invention.

On the contrary, it was observed that the colour varies from blue-tinted green to grey for the 10 coatings obtained with the comparative composition C₂.

The reproducibility of the colour of the coatings which are obtained with the composition C₁ according to the invention is thus much better than that of the coatings which are obtained with the comparative composition C₂.

1. Use, for the dyeing and/or making up of keratin materials, of at least one particle comprising:
(i) a core (1) constituted of at least one oxide of element M of formula (I): 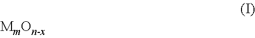
in which:
M represents an element chosen from the alkali metals of column 1, the alkaline earth metals of column 2 and the elements of columns 3 to 14 of the Periodic Table of the Elements, bismuth, selenium, tellurium and astatine,
m represents an integer greater than or equal to 1,
n represents an integer greater than or equal to 1,
x is equal to 0 or represents a non-integral number greater than 0 and strictly less than n, and
(ii) an upper coating layer (2), covering the surface of said core (1), constituted of at least one compound of formula (II): 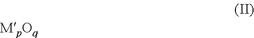
in which:
M′ is an element, other than M, chosen from selenium and the elements of columns 4, 13 and 14 of the Periodic Table of the Elements,
p represents an integer greater than or equal to 1,
q represents an integer greater than or equal to 0.

2. Use according to claim 1, characterized in that the element M is chosen from iron, zinc, aluminium, silicon, selenium, sodium, potassium, magnesium and calcium; preferably from iron, zinc and aluminium; and more preferentially from iron and zinc.

3. Use according to claim 1, characterized in that, taken together or separately:
m represents an integer ranging from 1 to 10, preferably from 1 to 5; and/or
n represents an integer ranging from 1 to 20, preferably from 2 to 10; and/or
p represents an integer ranging from 1 to 4, preferably equal to 1 or 2, more preferentially equal to 1; and/or
q represents an integer ranging from 0 to 4.

4. Use according to claim 1, characterized in that the oxide(s) of element M of formula (I) are chosen from: FeO, Fe₂O₃, Fe₃O₄, the compounds of formula FeO₁₋ₓ with x a non-integral number strictly of between 0 and 1, the compounds of formula Fe₃O₄₋ₓ with x a non-integral number strictly of between 0 and 4, the compounds of formula Fe₂O₃₋ₓ with x a non-integral number strictly of between 0 and 3, and the compounds of formula ZnO₁₋ₓ with x a non-integral number strictly of between 0 and 1.

5. Use according to claim 1, characterized in that the element M′ is chosen from selenium, titanium, aluminium and the elements of column 14 of the Periodic Table of the Elements; preferably from selenium, titanium, aluminium, carbon and silicon; more preferentially from carbon and silicon.

6. Use according to claim 1, characterized in that the compound(s) of formula (II) are chosen from carbon, SiO₂, SnO₂ and Al₂O₃, preferably from carbon and SiO₂.

7. Use according to claim 1, characterized in that the particle comprises:
(i) a core (1) constituted of an oxide of element M of formula (I) chosen from: FeO, Fe₂O₃, Fe₃O₄, the compounds of formula FeO₁₋ₓ with x a non-integral number strictly of between 0 and 1, the compounds of formula Fe₃O₄₋ₓ with x a non-integral number strictly of between 0 and 4, the compounds of formula Fe₂O₃₋ₓ with x a non-integral number strictly of between 0 and 3, and the compounds of formula ZnO₁₋ₓ with x a non-integral number strictly of between 0 and 1; and
(ii) an upper coating layer (2), covering the surface of said core (1), constituted of a compound of formula (II) chosen from carbon, SiO₂, SnO₂ and Al₂O₃.

8. Use according to claim 1, characterized in that the (M/M′) molar atomic ratio of the particle is strictly greater than 0.3; preferably greater than or equal to 1; more preferentially greater than or equal to 3; more preferentially included in the range extending from 3 to 10.

9. Use according to claim 1, characterized in that the number-average thickness dm of the upper coating layer (2), measured by transmission electron microscopy (TEM), is included in the range extending from 1 to 20 nm, preferably from 1 to 10 nm and more preferentially from 2 to 6 nm.

10. Use according to claim 1, characterized in that the number-average diameter of the particle, determined by transmission electron microscopy (TEM), is included in the range extending from 4 to 5000 nm, preferably from 10 to 3000 nm and more preferentially from 30 to 1000 nm.

11. Use according to claim 1, characterized in that the upper coating layer (2) of the particle covers at least 90% of the surface of the core (1), preferably covers the whole of the surface of the core (1).

12. Particle comprising:
(i) a core (1) constituted of at least one suboxide of element M of formula (I′): 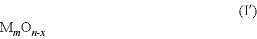
in which:
M represents an element chosen from the alkali metals of column 1, the alkaline earth metals of column 2 and the elements of columns 3 to 14 of the Periodic Table of the Elements, bismuth, selenium, tellurium and astatine; preferably M is chosen from iron, zinc, aluminium, silicon, selenium, sodium, potassium, magnesium and calcium; more preferentially from iron, zinc and aluminium; and more preferentially still from iron and zinc,
m represents an integer greater than or equal to 1,
n represents an integer greater than or equal to 1,
x represents a non-integral number greater than 0 and strictly less than n, and
(ii) an upper coating layer (2), covering the surface of said core (1), constituted of at least one compound of formula (II): 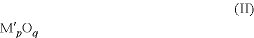
in which:
M′ is an element, other than M, chosen from selenium and the elements of columns 4, 13 and 14 of the Periodic Table of the Elements; preferably, M′ is chosen from selenium, titanium, aluminium and the elements of column 14 of the Periodic Table of the Elements; more preferentially from selenium, titanium, aluminium, carbon and silicon; more preferentially still from carbon and silicon,
p represents an integer greater than or equal to 1,
q represents an integer greater than or equal to 0.

13. Particle according claim 12, characterized in that the suboxide(s) of element M of formula (I′) are chosen from the compounds of formula FeO₁₋ₓ with x a non-integral number strictly of between 0 and 1, the compounds of formula Fe₃O₄₋ₓ with x a non-integral number strictly of between 0 and 4, the compounds of formula Fe₂O₃₋ₓ with x a non-integral number strictly of between 0 and 3, and the compounds of formula ZnO₁₋ₓ with x a non-integral number strictly of between 0 and 1.

14. Particle according to claim 12, characterized in that the (M/M′) molar atomic ratio of the particle is strictly greater than 0.3; preferably greater than or equal to 1; more preferentially greater than or equal to 3; more preferentially included in the range extending from 3 to 10.

15. Particle according to claim 12, characterized in that the upper coating layer (2) of the particle covers at least 90% of the surface of the core (1), preferably covers the whole of the surface of the core (1).

16. Composition, preferably cosmetic, comprising one or more particles as defined in claim 12.

17. Process for the preparation of one or more particles as defined in claim 1, comprising at least the following stages:
a. preparing a composition (A) by adding one or more precursors of element M to a combustible solvent or to a mixture of combustible solvents; then
b. forming a flame, in a flame spray pyrolysis device (10), by injecting the composition (A) and an oxygen-containing gas (G) until aggregates of oxide of element M are obtained; then
c. injecting a composition (B) comprising one or more precursors of element M′ until an upper coating layer (2) constituted of element M′ or of oxide of element M′ is obtained, at the surface of said aggregates of oxide of element M;
said element M being chosen from the alkali metals of column 1, the alkaline earth metals of column 2 and the elements of columns 3 to 14 of the Periodic Table of the Elements, bismuth, selenium, tellurium and astatine; preferably M being chosen from iron, zinc, aluminium, silicon, selenium, sodium, potassium, magnesium and calcium; more preferentially from iron, zinc and aluminium; and more preferentially still from iron and zinc; and
said element M′ being other than M and chosen from selenium and the elements of columns 4, 13 and 14 of the Periodic Table of the Elements; preferably, M′ being chosen from selenium, titanium, aluminium and the elements of column 14 of the Periodic Table of the Elements; more preferentially from selenium, titanium, aluminium, carbon and silicon; more preferentially still from carbon and silicon; and
said flame spray pyrolysis device (10) being isolated from the exterior air, so that the amount of oxygen present in said device is controlled.

18. Process according to claim 17, characterized in that, taken together or separately:
the (M/M′)_{injected} molar atomic ratio is greater than or equal to 0.25; preferably included in the range extending from 0.25 to 120; more preferentially from 0.25 to 99; more preferentially still from 1 to 80; and better still from 3 to 20; and/or
stage b. is carried out in a first chamber (20) of a flame spray pyrolysis device (10) and stage c. is carried out in a second chamber (30) of the device (10) in fluidic communication with the first chamber (20); and/or
the composition (B) comprises one or more solvents; preferably the solvent(s) are chosen from polar protic solvents other than water; more preferentially from (C₁-C₈)alkanols; and better still the solvent is ethanol; and/or
the molar amount of oxygen-containing gas (G)_{injected} during stage b. is strictly less than the molar amount of oxygen-containing gas necessary to cause the composition (A) to react with the oxygen in a stoichiometric ratio.

19. Process according to claim 17, characterized in that the flame spray pyrolysis device (10) is pressurized by an inert gas (G2) preferably chosen from nitrogen, methane, argon, hydrogen, hydrogen sulfide and ammonia; more preferentially from nitrogen, methane, hydrogen and argon; more preferentially still from nitrogen and argon, and better still by nitrogen.

20. A particle, characterized in that it is obtained by the process as defined in claim 17.

21. Flame spray pyrolysis device (10) comprising a first chamber (20), a second chamber (30) connected fluidically to the first chamber, and an injection system (40) comprising a first feed (42) emerging in the first chamber (20) and capable of delivering a first composition (A) and a first oxygen-containing gas (G), and a second feed (44) emerging in the first chamber (20) and capable of delivering a mixture (P) comprising oxygen and one or more combustible gases, the first and second feeds (42, 44) being separate from one another, said device (10) comprising a third feed (38) capable of delivering a second composition (B) comprising one or more precursors of element M′ into the second chamber (30), the first and second chambers (20, 30) of said device (10) being isolated from the external air, so that the amount of oxygen present in said first and second chambers (20, 30) is controlled.

22. Device (10) according to claim 21, in which the first and second feeds (42, 44) are coaxial, the second feed (44) at least partially surrounding the first feed (42).

23. Device (10) according to claim 21, in which the first chamber (20) comprises two separate compartments (22, 24), the first compartment (22) comprising a first opening (22a) in which the injection system (40) emerges and a second opening (22b), on the side opposite the first opening (22a), the second compartment (24) surrounding the first compartment (22) and being isolated from the external air, said second compartment (24) being separated from the first compartment (22) by a first partition (26).

24. Device (10) according to claim 23, comprising an injector (28) configured to inject a second gas (G2) into the second compartment (24) of the first chamber (20), the first partition (26) being permeable to said second gas (G2).

25. Device (10) according to claim 23, in which the second chamber (30) comprises two separate compartments (32, 34), the first compartment (32) comprising a first opening (32a) in fluidic communication with the second opening (22b) of the first chamber (20) and a second opening (32b), on the side opposite the first opening (32a), the second compartment (34) surrounding the first compartment (32) and being isolated from the external air, said second compartment (34) being separated from the first compartment (32) by a second partition (36) and having a feed (38) for feeding the second composition (B) into the second chamber (30).

26. Device (10) according to claim 25, comprising an additional feed (38) configured to inject a third gas (G3) into the second compartment (34) of the second chamber (30), the second partition (36) being permeable to said third gas (G3).

27. Device (10) according to claim 21, further comprising a collecting system (50) positioned above the second chamber (30) and permeable to gases.
